(19) United States
(12) Patent Application Publication
(10) Pub. No.: US 2007/0028930 A1
(43) Pub. Date: Feb. 8, 2007

(54) Active agent and formulations to minimize or alleviate bladder urgency and irritation and/or to enhance sexual function

(75) Inventors: Michael B. Chancellor, Pittsburgh, PA (US); Pradeep Tyagi, Pittsburgh, PA (US); Jonathan Kaufman, Pittsburgh, PA (US)

Correspondence Address: THE WEBB LAW FIRM, P.C., 700 KOPPERS BUILDING, 436 SEVENTH AVENUE, PITTSBURGH, PA, 15219, US

(73) Assignee: Delithe Natural Products Inc., Pittsburgh, PA (US)

(21) Appl. No.: 11/475,834

(22) Filed: Jun. 27, 2006

Related U.S. Application Data
(60) Provisional application No. 60/694,215, filed on Jun. 27, 2005.
(60) Provisional application No. 60/801,932, filed on May 19, 2006.

(51) Int. Cl. A61B 19/00

(52) U.S. Cl. 128/898

(57) ABSTRACT

To treat premature ejaculation, delay ejaculation, or improve male sexual control, control bladder urgency and/or minimize or alleviate irritated bladder or bladder urgency, an effective amount of St. John's Wort extract rich in hyperforin is administered to a male prior to sexual intercourse, or to a male or female patient otherwise in need thereof.

### CROSS REFERENCE TO RELATED APPLICATIONS

This application claims the benefit of U.S. Provisional Application Ser. No. 60/694,215, filed Jun. 27, 2005, and U.S. Provisional Application Ser. No. 60/801,932, filed May 19, 2006, both of which are herein incorporated by reference in their entirety.

### BACKGROUND OF THE INVENTION

1. Field of the Invention

The invention pertains to active agents intended for urological health and enhancement of male sexual performance. Plant extracts rich in certain natural neurotransmitter reuptake inhibitors can be used for prolonging male sexual activity (delaying ejaculatory response) as well as relieving bladder irritation and reducing urinary urgency.

2. Introduction

Treatment of premature ejaculation or enhancement of male sexual control is usually achieved in the clinic by psychological therapies, local anesthetics, or with the use of devices. All of these treatments have significant drawbacks. Psychological therapies benefit only a subset of patients, require intensive counseling, and may not be affordable to all or even many patients. Furthermore, psychological therapies cannot alleviate premature ejaculation resulting from non-psychological causes. Anesthetic agents decrease sensitivity of the penile shaft, and while these help some people they do so at the cost of diminishing sexual pleasure. Devices, moreover, can be awkward, inconvenient and embarrassing to use.

Treatment of premature ejaculation or enhancement of male sexual control is frequently enhanced in the clinic by the off-label chronic use of selective serotonin (5-HT) reuptake inhibitors (SSRIs) or antidepressant drugs such as Fluoxetine, Sertraline, and Paroxetine. (See U.S. Pat. Nos. 4,507,323, 4,940,731, 5,151,448, and 5,276,042 and Rosen et al., J. Clin. Psychopharmacol., 19, 67-85 (1999).) SSRIs have produced significant success in treating depression and related illnesses and have become among the most prescribed drugs. The majority of patients who take these drugs experience effects on their sexual function. SSRIs and antidepressants that augment 5-HT function have, overall, relatively more effect on sexual function. SSRIs nonetheless have a slow onset of action, often taking several weeks to produce their full therapeutic effect. SSRIs are believed to work by blocking the neuronal reuptake of serotonin, thereby increasing the concentration of serotonin in the synaptic space, and thus increasing the activation of postsynaptic serotonin receptors. However, although a single dose of an SSRI can inhibit the neuronal serotonin transporter which would be expected to increase synaptic serotonin, a favorable absorption profile is needed for SSRI or similar drugs to enhance male sexual control.

The process of ejaculation requires two sequentially distinct motor actions, namely, emission and expulsion to eject seminal fluid from the urethral meatus. The emission phase is the first one to happen and it involves deposition of seminal fluid from the ampullary vas deferens, seminal vesicles and prostate gland into the posterior urethra. The second phase is the expulsion of semen which involves closure of the bladder neck followed by the characteristic rhythmic contractions of the proximal urethra by pelvi-perineal striated muscles with a primary role for the bulbospongiosus BS muscle and intermittent relaxation of external urethral sphincter. BS muscle activity is responsible for the propulsion of semen from the prostatic urethra to the urethral meatus as well as its forceful expulsion. Ejaculation is a spinal sympathetic reflex generated in the lumbosacral spinal cord and it requires coordination of neural peripheral autonomic and somatic efferent inputs from hypogastric, pelvic and pudendal nerves following appropriate central and peripheral stimuli.

Sympathetic motor neurons control the emission phase of ejaculation reflex and the propulsion phase is executed by somatic and autonomic motor neurons. These motor neurons are located in the thoracolumbar and lumbosacral spinal cord and are activated in a coordinated manner when sufficient sensory input to reach the ejaculatory threshold has entered the central nervous system. Briefly, the interneurons in the lumbar spinal cord and supraspinal areas of CNS are believed to modulate the ejaculatory threshold using 5-HT as a neurotransmitter.

The effects of SSRIs and tricyclic antidepressants (TCA) on transport and emission of spermatozoa may depend on alterations in the contractility of vas deferens smooth muscle. However, these antidepressants have limited efficacy in treating premature ejaculation and improving male sexual control, and they can cause troublesome side effects in individuals, such as potential suicide risk. Disease states or adverse interactions with other drugs may contraindicate the use of these drugs, moreover.

The background knowledge regarding efficacy of the present composition in painful bladder syndrome is based on the following concepts. Central neural control of the bladder at the spinal cord level is influenced by pudendal somatic motor nucleus, innervated by serotonin and norepinephrine emitting neurons. Central modulation of the lower urinary tract activity is possible with agonists or antagonists of serotonin and norepinephrine. The inhibitory circuit of the descending bulbospinal pathway to bladder and pudendal nerves uses serotonin. Electrical stimulation of serotonin neurons and activation of postsynaptic serotonin receptors in the spinal cord causes inhibition of bladder contractions in cats (Espey and Downie, 1995).

It is generally acknowledged that chronic administration of hyperforin, the active ingredient of St. John's Wort, evokes a pharmacological action similar to antidepressant drugs such as Imipramine, Fluoxetine, Sertraline and other congeners of same pharmacological category. St. John's Wort extract caused a 50% inhibition of serotonin reuptake in rat synaptosomes, and also showed equal affinity for inhibiting dopamine and norepinephrine, in certain studies. St. John's Wort extract is rich in hyperforin and inhibits excitatory transmission of rat bladder strips and directly inhibits smooth muscle contractility. St. John's Wort has been known to inhibit synaptosomal activation of sympathetic and somatic nervous systems that leads to inhibition of bladder contraction.

The term premature ejaculation as used herein means a situation where male has a limited amount of control over the timing of his ejaculation such that he or his sexual partner have a desire to prolong his ejaculatory response.

### SUMMARY OF THE INVENTION

To treat premature ejaculation, delay ejaculation, or improve male sexual control, an effective amount of St. John's Wort extract rich in hyperforin is administered to a male prior to sexual intercourse. By an effective amount is meant a nontoxic, but sufficient, amount of a St. John's Wort extract rich in hyperforin to delay ejaculation. Effective dosage forms, modes and times of administration, and dosage amounts may be determined empirically, and making such determinations is within the skill of the art. Preferred is a single dose taken orally before sexual intercourse. In particular, it has been found that an effective dosage of St. John's Wort extract rich in hyperforin and effective to delay ejaculation contains pure hyperforin in the amount of from about 5 to 250 milligrams (mg), more preferably 10 to about 200 mg, more preferably from about 15 to about 150 mg, even more preferably about 25-75, and most preferably about 50 mg, administered sublingually or buccally within about 30 to about 60 minutes prior to sexual intercourse. However, it is understood by those skilled in the art that the dosage amount will vary with the particular form of St. John's Wort extract employed, the route(s) of administration, the timing of the administration, the identity of any other drugs being administered, whether or not the male suffers from premature ejaculation or lack of sexual control and the severity of the condition, the age, size and condition of the patient, and like factors known in the medical art. In general, a suitable dose will be that amount of the compound which is the lowest dose effective to improve sexual control without toxicity.

### BRIEF DESCRIPTION OF THE DRAWING(S)

FIG. 1 shows EMG tracings of rat following rises in urethral pressure and those after injection of St. John's Wort extract rich in hyperforin or its vehicle. EMG recording of BS muscle of male rat under anesthesia documented sudden rises in urethral pressure. Notice the lack of latency in bursts of EMG with rise in urethral pressure in this model. Urethral pressure spikes are a result of antegrade infusions of saline for 2 sec duration. Reflex EMG activity in vehicle-treated rats follows rise in urethral pressure induced by infusion of saline into prostatic urethra. Only limited EMG activity occurs in response to identical stimulus in treated rats.

FIG. 2 shows CMG tracing of normal unirritated rat bladder after injection of St. John's Wort extract rich in hyperforin. Extract is injected in intraperitoneum at a time indicated by arrows at approximately 60 min.; data for two rats are shown.

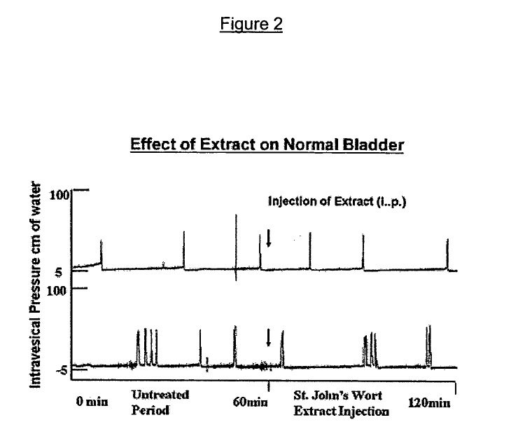

FIG. 3 shows CMG tracing of irritated bladder after injection of St. John's Wort extract rich in hyperforin. There was a 83.5±5.5% decrease in ICI (average intercontraction interval) of control groups injected with vehicle after acetic acid infusion (n=6). Rats injected with St. John's Wort Extract showed a significantly decreased response (ICI decrease) of only 34.6±11.1%, n=6) after acetic acid infusion (p<0.005). Amplitude of contraction and pressure threshold between control and drug-treated rats showed no statistical significance (p>0.05). Acute administration of St. John's Wort Extract can suppress nociceptive responses induced by bladder irritation. Acetic acid was administered to bladder lumen at times indicated by arrows, approximately 60 min. Data for four rats are shown.

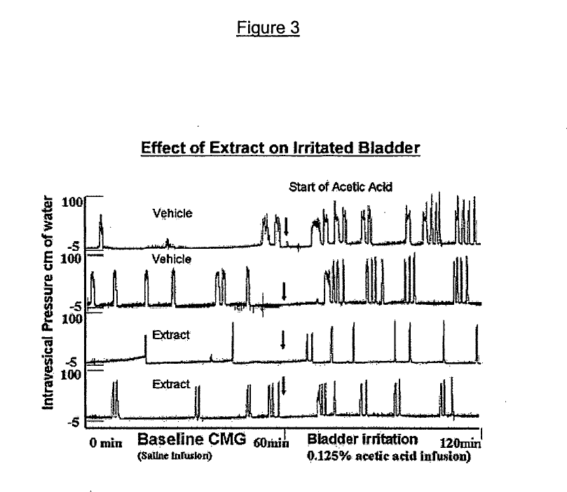

FIG. 4 shows the serum kinetics of sublingual formulation and traditional St. John's Wort capsule taken orally by a healthy male volunteer on separate occasions. The St. John's Wort capsule was swallowed immediately, while the sublingual formulation was permitted to dissolve in the mouth prior to swallowing. Blood samples were obtained prior to and at serial time points after the supplement administrations. Hyperforin content was measured in blood samples via liquid chromatography and mass spectrometry. The sublingual formulation induced significant serum levels of hyperforin within 30 minutes of administration, and a peak level at approximately 60 minutes. The St. John's Wort capsule required approximately two hours to induce comparable levels of hyperforin, and approximately four hours to reach a peak level.

FIG. 5 shows (vertical axis) intravaginal ejaculatory latency time (IELT) in minutes; horizontal axis is number of days elapsed during the study. Male subject had sexual intercourse with his partner seven times—three times prior to using hyperforin-rich extract, and four times after using hyperforin-rich extract beginning after the ninth day in study. IELT was measured by the partner using a stopwatch, and significantly increased with use of hyperforin-rich extract.

### DETAILED DESCRIPTION OF THE INVENTION

The St. John's Wort extract rich in hyperforin material may be administered by any suitable route of administration, including orally, nasally, rectally, parenterally (e.g., intravenously, subcutaneously, or intramuscularly), topically (i.e., delivery to the skin or mucosa), transdermally (i.e., delivery by passage of a drug through the skin into the bloodstream), or transmucosally (i.e., delivery by passage of a drug through the mucosal tissue into the bloodstream). Most preferred is sublingual administration; buccal administration is also among the preferred embodiments. Overall, then, the transmucosal route of administration is important.

Any conventional technique may be used for the preparation of pharmaceutical composition or nutritional supplement according to the invention. The active ingredient may be contained in a formulation that provides quick release, after administration to the individual. The nutritional supplement of the invention may be prepared, packaged, or sold as a single unit dose, or as a plurality of single unit doses. As used herein, a unit dose is a discrete amount of the nutritional supplement composition comprising a predetermined amount of the active ingredient. The amount of the active ingredient in each unit dose is generally equal to the total amount of the active ingredient which would be administered.

A formulation of a pharmaceutical composition of the invention suitable for sublingual administration may in the form of a discrete solid dosage unit or a fraction thereof. Solid dosage units include, for example, a tablet, mouthwash, pocketpak strips using instant dissolving strip technology, toothpaste, a cachet, a troche, or a lozenge. Each solid dosage unit contains a predetermined amount of the active ingredient, for example a unit dose or fraction thereof. Other formulations suitable for administration include, but are not limited to, a powdered or granular formulation, an aqueous or oily suspension, an aqueous or oily solution, or an emulsion. As used herein, an oily liquid is one which comprises a carbon or silicon based liquid that is less polar than water. Preferred is a lozenge or rapid dissolving tablet.

Lozenges are solid dosage forms intended to be placed under tongue for systemic absorption. In solid dosage forms of the invention for oral administration (lozenges rapid dissolving tablets and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise (11) buffering agents or (12) antioxidants such ascorbic acid to prevent oxidation of hyperforin. These compositions may also optionally contain opacifying agents and antioxidants such as ascorbic acid and sodium metabisulfite to improve the stability of hyperforin in formulation.

A tablet comprising the active ingredient may be made, for example, by compressing or molding the active ingredient, optionally containing one or more additional components. Compressed tablets may be prepared by compressing, in a suitable device, the active ingredient in a free-flowing form such as a powder or granular preparation, optionally mixed with one or more of a binder, a lubricant, a glidant, an excipient, a surface active agent, and a dispersing agent. Molded tablets may be made by molding, in a suitable device, a mixture of the active ingredient, a pharmaceutically acceptable carrier, and at least sufficient liquid to moisten the mixture.

A major drawback of lozenges is the requirement of low drug loading capacity. In the disclosed invention the St. John's Wort extract rich in hyperforin, i.e., 5-250 mg hyperforin, will be formulated into a troche that slowly dissolves under the tongue following oral administration. In the present invention we disclose the dose of St. John's Wort extract rich in hyperforin that can be loaded into an easy to administer small sized troche for sublingual administration.

Although it is possible for the St. John's Wort extract rich in hyperforin to be administered alone, it is preferable to administer it as a pharmaceutical formulation (composition). The pharmaceutical compositions will comprise a St. John's Wort extract rich in hyperforin as the active ingredient in admixture with one or more pharmaceutically-acceptable carriers and, optionally, with one or more other compounds, drugs, or other materials. Each carrier must be acceptable in the sense of being compatible with the other ingredients of the formulation and not injurious to the male who will take the composition. Pharmaceutically-acceptable carriers are well known in the art. Regardless of the route of administration selected, the active ingredients are formulated into pharmaceutically-acceptable dosage forms by conventional methods known to those of skill in the art.

Other possible formulations of the invention suitable for oral administration may be in the form of cachets, pocketpak strips, pills, tablets, powders, granules or as a solution or a suspension in an aqueous or non-aqueous liquid, or an oil-in-water or water-in-oil liquid emulsions, or as an elixir or syrup and the like, each containing a predetermined amount of the active ingredient. Preferred oral administration forms are tablets and capsules.

Liquid dosage forms for oral administration of the compounds of the invention include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming, thickening, and preservative agents.

Suspensions, in addition to the active ingredient, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

St. John's Wort extract rich in hyperforin, including pure hyperforin, can be specially formulated, for example, as a rapid oral release formulation which can allow rapid buildup of higher serum levels of drug following administration. Chronic administration of St. John's Wort extract rich in hyperforin, including water insoluble hyperforin itself, can be specially formulated into an aqueous concocted drink which can achieve rapid serum level and its daily administration can evoke and sustain a health benefit. The Formulation of St. John's Wort extract helps men with poor sexual control. This can also delay ejaculation time and improve male sexual control.

St. John's Wort extract rich in hyperforin, used herein, can be formulated into a rapid release formulation for sublingual administration prior to sexual act. The St. John's Wort extract rich in hyperforin can be prepared from ethanolic extract of St. John's Wort plant (or by raw plan material) by using carbon dioxide extraction or supercritical carbon dioxide extraction.

All preparations and delivery of St. John's Wort extracts rich in hyperforin can be used for the treatment of bladder irritation as well as premature ejaculation and/or enhancement of male sexual function. The preparations can be used for reducing urinary urgency as well, even in the absence of bladder irritation.

Sublingual administration allows the hyperforin to escape the hepatic first pass metabolism and affords rapid rise in serum levels of drugs such as nitrates used in treatment of angina. In general, the disclosed method include formulating the dietary supplement of St. John's Wort extract rich in hyperforin or its bioactive ingredient hyperforin itself into a rapid release dosage form such as lozenge or troche which can improve the health benefits derived from this dietary supplement.

Hyperforin is a relatively unstable molecule, and in particular subject to oxidation. The decay rate of hyperforin is increased by exposure to increased temperatures and exposure to light. This instability makes the marketing of hyperforin rich products difficult. Our preferred formulation of hyperforin as a dietary supplement or pharmaceutical composition involves packaging each dosage individually with nitrogen or carbon dioxide gas such that the dosages are not exposed to ambient oxygen (other non-oxygen or non-oxidizing gases can be substituted), including an antioxidant in the hyperforin-rich tablet formulation, using opaque packaging materials such that the formulation is not exposed to light until ready to use, keeping inventory cold prior to distribution either with liquid nitrogen or a laboratory or commercial freezer, or some or all of the above.

Delivery of pure hyperforin or St. John's Wort extract rich in hyperforin in a rapid release formulation in the mouth can achieve its rapid onset of action with favorable serum kinetics. These improved serum kinetics make it possible for the formulations to be used on-demand rather than chronically, which is favorable for example when using in male sexual control and for the relief of temporary flare-up associated with interstitial cystitis.

Pharmaceutical tablets or dietary supplements that are designed to dissolve in the mouth are rarely a black, dark brown, or dark green color inasmuch as such dark coloring is undesirable. Common St. John's Wort extract, conducted with ethanol, produces a dark brown powder, which is a color unsuitable commercially for an oral rapid dissolve formulation because it discolors the mouth. A carbon dioxide extraction done on this dark powder produces a translucent yellowish green paste that is a commercially suitable color for a rapid dissolve tablet. Thus a preferred form of the hyperforin-rich plant extract is a carbon dioxide extract. It is also possible to extract the St. John's Wort with ethanol (or a suitable lower alcohol or other organic solvent) and then with supercritical carbon dioxide, or with supercritical carbon dioxide first and then ethanol. The advantage of the double extraction is that hyperforin is soluble in both ethanol and supercritical carbon dioxide, so the “double” extraction reduces or eliminates any impurities that are not likewise soluble in both supercritical carbon dioxide and ethanol. Notwithstanding the ability to use solvents other than ethanol and supercritical carbon dioxide, these two solvents in particular are completely pharmaceutically benign and create no solvent-trace problems or toxicities.

### EXAMPLE 1

Male rats are frequently used for modeling clinical symptoms of premature ejaculation because their intromissions and ejaculations are clearly discemable and can be pharmacologically evoked. In addition, both rats and humans have an average ejaculation latency of about five minutes, although large individual differences exist within both human and rat populations. Techniques have recently been developed to induce male rat ejaculation on-demand, and thus provide an animal model that does not involve the observation and assessment of rat sexual behavior. One such well accepted model of ejaculation is based on urethro-genital (UG) reflex method that relies on measuring the expulsion phase of ejaculation. The bulbospongious (BS) muscle at the base of the penis and the bulbocavemosus muscle surrounding the urethral bulb are the key muscles involved in the expulsion phase of ejaculation for ejecting semen or urethral contents. The contractile activity of BS muscles is used as surrogate marker for expulsion phase in anesthetized rats by persons skilled in the art.

Rat ejaculation model for developing this invention was based on UG reflex, which involves inducing rhythmic BS muscle contraction by abrupt rise in urethral pressure. Under urethane anesthesia, a catheter was inserted into prostatic urthera after laprotomy and wire electrodes were placed in BS muscles for electromyogram (EMG). Intracerebroventricular (i.c.v) injection of 8OH-DPAT (60 μg) was made into the lateral cerebral ventricle of the brain. St. John's Wort extract rich in hyperforin was injected intraperitoneally one hour prior to injection of 8OH-DPAT. Sudden rises in urethral pressure were achieved by pushing saline at the rate of 116 μl/sec for 2 seconds in prostatic urethra to cause rhythmic contraction of BS muscles. Upon i.c.v. injection of 8OH-DPAT, in the saline treated rats, BS muscle contraction was seen in absence of rise in urethral pressure and frequency of waves was increased with concomitant rise in urethral pressure. Prior administration of St. John's Wort extract rich in hyperforin abolished the spontaneous muscle activity induced by 8OH-DPAT and reduced the EMG frequency of BS muscles following rise in urethral pressure.

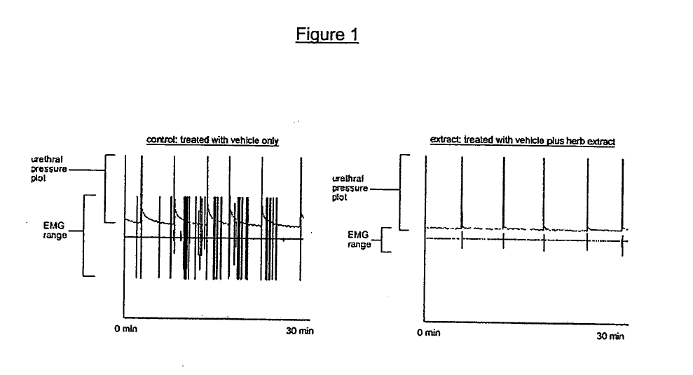

The extract was prepared from powdered plant using supercritical carbon dioxide as an eluent. Control rats were injected with vehicle alone (FIG. 1). Vehicle treated rats showed multiple bursts in electrical activity of BS muscle even in the absence of rise in urethral pressure, and upon i.c.v. injection of 8OH-DPAT both amplitude and frequency of bursts was increased with accompanying rise in urethral pressure. Prior administration of St. John's Wort extract rich in hyperforin abolished the spontaneous muscle activity induced by 8OH-DPAT and attenuated the frequency and amplitude in EMG of BS muscles accompanying with the rise in urethral pressure. St. John's Wort extract rich in hyperforin can blunt the pro-ejaculatory effect of 8OH-DPAT in rat.

### EXAMPLE 2

Female Sprague-Dawley rats anesthetized with urethane (1.2 g/kg). Transurethral cystometrogram CMG was done by infusing saline (0.04 ml/min). Baseline CMG was followed by infusion of 0.125% acetic acid. St. John's Wort Extract rich in hyperforin (10 mg/kg dissolved in 30% cremophor in saline, keeping volume of injection of 0.3 mL) was injected (i.p.) at the dose of 10 mg/kg one hour prior to acetic acid infusion. Control rats were injected with 0.3 mL of vehicle (previous, in vivo bladder studies in rats showed 10 mg/kg dose of SSRI (Duloxetine) as effective (Thor and Katofiasc, 1995)). Intercontraction interval (ICI) calculated as time difference between two peaks, Amplitude of Contraction, and Pressure Threshold were measured for each rat. Decrease in ICI for each rat by acetic acid over its baseline value was calculated as a percent reduction.

### EXAMPLE 3

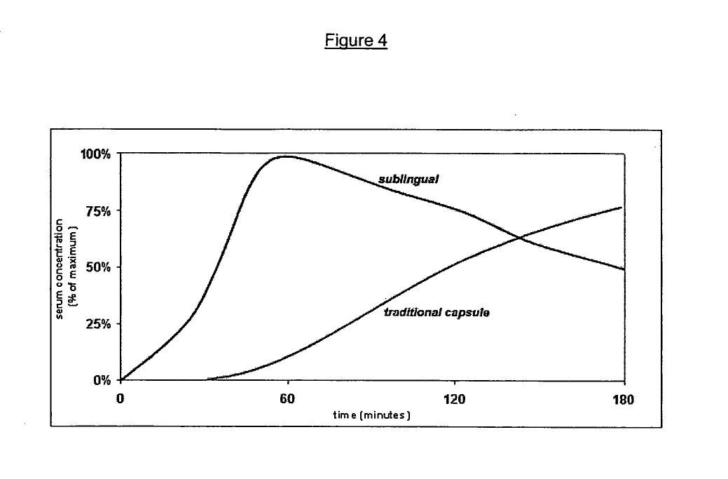

The sublingual formulation and traditional St. John's Wort capsule were taken orally by a healthy male volunteer on separate occasions. The St. John's Wort capsule was swallowed immediately, while on a different occasion the sublingual formulation was permitted to dissolve in mouth prior to swallowing. Blood samples were obtained prior to and at serial time points after the supplement administrations. Hyperforin content was measured in blood samples via liquid chromatography and mass spectrometry. The sublingual formulation induced significant serum levels of hyperforin within 30 minutes of administration, and a peak level at approximately 60 minutes. The St. John's Wort capsule required approximately two hours to induce comparable levels of hyperforin, and approximately four hours to reach a peak level. See FIG. 4.

### EXAMPLE 4

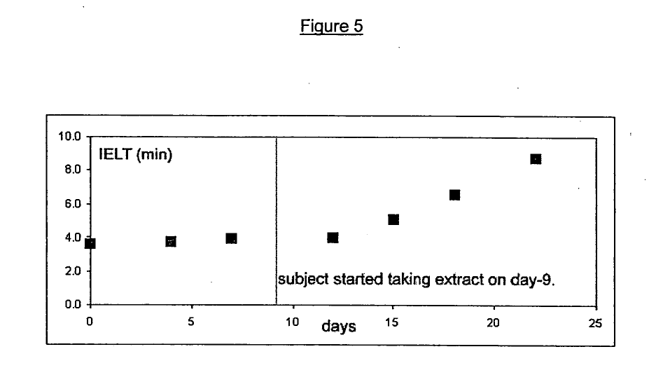

A male subject had sexual intercourse with his partner seven times within the study, three times prior to using hyperforin-rich extract, and four times after using hyperforin-rich extract—beginning after the ninth day in study. IELT was measured by the partner using a stopwatch and significantly increased with use of the present hyperforin-rich extract. See FIG. 5.

### EXAMPLE 5

Formula for Single Lozenge of St. John's Wort: St. John's Wort's extract (standardized to 30 % Hyperforin) 83.3 mg, Xylitol 1100 mg, Mannitol 200 mg, Ascorbic acid 5 mg, Citric acid 15 mg, Povidone-VA-64 70 mg, Lake Yellow 12 mg, Ammonium glycyrrhizinate 15 mg, Lime Flavor spray dried 5 mg, Orange oil spray dried 5 mg, Hydrogenated vegetable oil 25 mg, Titanium dioxide 10 mg, Stearic acid Powder 15 mg. Preparation: manufacturing by direct compression while avoiding exposure to either moisture or light. Mannitol and Povidone VA 64 were mixed with Hyperforin extract to prepare the drug mixture. The drug mixture was mixed with diluent xylitol sieved through a 20 mesh sieve and mixed with other ingredients. The complete powder mixture was sieved through a 50 mesh sieve and then compressed with a suitable tablet press using a square punch with greater than 14 mm to prepare a dosing of 1610 per lozenge. Packaging: Blister packaging under a steady stream of nitrogen.

The invention claimed is:

1. A method of improving male sexual control, comprising administering, in unit dosage form, at least one dose of a St. John's Wort extract rich in hyperforin to a male in need of treatment, wherein said dosage form contains about 5-250 mg hyperforin together with at least one pharmaceutically acceptable excipient, wherein the administering is conducted within sixty minutes prior to sexual activity.

2. The method according to claim 1 wherein said dosage form contains about 10-200 mg hyperforin.

3. The method according to claim 1 wherein said dosage form contains about 15-150 mg hyperforin.

4. The method according to claim 1 wherein said dosage form contains about 25-75 mg hyperforin.

5. The method according to claim 1 wherein said dosage form contains about 50 mg hyperforin.

6. The method according to claim 1 wherein said dosage form contains St. John's Wort extract rich in hyperforin and wherein said extract is prepared by carbon dioxide extraction.

7. The method according to claim 1 wherein said dosage form contains St. John's Wort extract rich in hyperforin and wherein said extract is prepared using supercritical carbon dioxide.

8. The method according to claim 1 wherein said dosage form contains St. John's Wort extract rich in hyperforin and wherein said extract is prepared using ethanol.

9. The method according to claim 1 wherein said dosage form contains St. John's Wort extract rich in hyperforin and wherein said extract is prepared using an ethanol extraction of the St. John's Wort and a supercritical carbon dioxide extraction of the extract so produced, or by using a supercritical carbon dioxide extraction of the St. John's Wort followed by an ethanol extraction of the extract so produced.

10. The method according to claim 1 wherein said St. John's Wort extract rich in hyperforin is prepareding using formulation, extraction, storage and packaging techniques which reduce oxidation.

11. The method according to claim 1 wherein the unit dosage form is selected from the group consisting of tablets, sublingual tablets, buccal tablets, capsules, troches, lozenges, cachets, pocketpak strips, pills, powders, granules, solutions, suspensions, elixirs, emulsions, microemulsions, ointments, and gels.

12. The method according to claim 1 wherein the unit dosage form is a tablet or capsule.

13. The method according to claim 1 wherein the unit dosage form contains a rapid dissolving agent.

14. A method for reducing bladder urgency in a male or female animal or patient by administering, in unit dosage form, at least one dose of a St. John's Wort extract rich in hyperforin to a male or female in need of such treatment, wherein said dosage form contains about 5-250 hyperforin together with at least one pharmaceutically acceptable excipient.

15. The method according to claim 14 wherein said dosage form contains about 10-200 mg hyperforin.

16. The method according to claim 14 wherein said dosage form contains about 15-150 mg hyperforin.

17. The method according to claim 14 wherein said dosage form contains about 25-75 mg hyperforin.

18. The method according to claim 14 wherein said dosage form contains about 50 mg hyperforin.

19. The method according to claim 14 wherein said dosage form contains St. John's Wort extract rich in hyperforin and wherein said extract is prepared by carbon dioxide extraction.

20. The method according to claim 14 wherein said dosage form contains St. John's Wort extract rich in hyperforin and wherein said extract is prepared using supercritical carbon dioxide.

21. The method according to claim 14 wherein said dosage form contains St. John's Wort extract rich in hyperforin and wherein said extract is prepared using ethanol.

22. The method according to claim 14 wherein said dosage form contains St. John's Wort extract rich in hyperforin and wherein said extract is prepared using an ethanol extraction of the St. John's Wort and a supercritical carbon dioxide extraction of the extract so produced, or by using a supercritical carbon dioxide extraction of the St. John's Wort followed by an ethanol extraction of the extract so produced.

23. The method according to claim 14 wherein said St. John's Wort extract rich in hyperforin is prepareding using formulation, extraction, storage and packaging techniques which reduce oxidation.

24. The method according to claim 14 wherein the unit dosage form is selected from the group consisting of tablets, sublingual tablets, buccal tablets, capsules, troches, lozenges, cachets, pocketpak strips, pills, powders, granules, solutions, suspensions, elixirs, emulsions, microemulsions, ointments, and gels.

25. The method according to claim 14 wherein the unit dosage form is a tablet or capsule.

26. The method according to claim 14 wherein the unit dosage form contains a rapid dissolving agent.

27. A method of minimizing or alleviating the symptoms of irritated bladder in a male or female patient, comprising administering, in unit dosage form, at least one dose of a St. John's Wort extract rich in hyperforin to a male or female in need of treatment, wherein said dosage form contains about 5-250 mg hyperforin together with at least one pharmaceutically acceptable excipient.

28. The method according to claim 27 wherein said dosage form contains about 10-200 mg hyperforin.

29. The method according to claim 27 wherein said dosage form contains about 15-150 mg hyperforin.

30. The method according to claim 27 wherein said dosage form contains about 25-75 mg hyperforin.

31. The method according to claim 27 wherein said dosage form contains about 50 mg hyperforin.

32. The method according to claim 27 wherein said dosage form contains St. John's Wort extract rich in hyperforin and wherein said extract is prepared by carbon dioxide extraction.

33. The method according to claim 27 wherein said dosage form contains St. John's Wort extract rich in hyperforin and wherein said extract is prepared using supercritical carbon dioxide.

34. The method according to claim 27 wherein said dosage form contains St. John's Wort extract rich in hyperforin and wherein said extract is prepared using ethanol.

35. The method according to claim 27 wherein said dosage form contains St. John's Wort extract rich in hyperforin and wherein said extract is prepared using an ethanol extraction of the St. John's Wort and a supercritical carbon dioxide extraction of the extract so produced, or by using a supercritical carbon dioxide extraction of the St. John's Wort followed by an ethanol extraction of the extract so produced.

36. The method according to claim 27 wherein said St. John's Wort extract rich in hyperforin is prepareding using formulation, extraction, storage and packaging techniques which reduce oxidation.

37. The method according to claim 27 wherein the unit dosage form is selected from the group consisting of tablets, sublingual tablets, buccal tablets, capsules, troches, lozenges, cachets, pocketpak strips, pills, powders, granules, solutions, suspensions, elixirs, emulsions, microemulsions, ointments, and gels.

38. The method according to claim 27 wherein the unit dosage form is a tablet or capsule.

39. The method according to claim 27 wherein the unit dosage form contains a rapid dissolving agent.
